(19) United States
(12) Patent Application Publication
(10) Pub. No.: US 2008/0099331 A1
(43) Pub. Date: May 1, 2008

(54) SOLID-STATE UREA BIOSENSOR AND ITS DATA ACQUISITION SYSTEM

(75) Inventors: Shen-Kan Hsiung, Jungli City (TW); Jung-Chuan Chou, Douliou City (TW); Tai-Ping Sun, Jhongli City (TW); Chung-We Pan, Pingtung County (TW)

Correspondence Address: PAI PATENT & TRADEMARK LAW FIRM, 1001 FOURTH AVENUE, SUITE 3200, SEATTLE, WA, 98154, US

(73) Assignee: CHUNG YUAN CHRISTIAN UNIVERSITY, Jhongli City (TW)

(21) Appl. No.: 11/306,809

(22) Filed: Jan. 12, 2006

(51) Int. Cl. G01N 33/487; G01N 27/26

(52) U.S. Cl. 204/403.01; 204/407

(57) ABSTRACT

The invention provides a method of fabrication of a solid-state urea biosensor and its data acquisition system. The biosensor includes a substrate, and three individual sensing areas separated by an insulating layer on the substrate. Each individual sensing area contains a conductive layer on the substrate, and a pH sensitive membrane is deposited thereon. An enzyme layer is deposited on one of the pH sensitive membranes to form a working electrode. The other two sensing areas are a quasi-reference electrode and a contrast electrode, respectively. The sensing signals are transferred to an instrumentation amplifier by conductive wires. The sensing signals are then transferred to a low-pass filter to reduce the high frequency noise. A data acquisition card is employed to transfer the sensing signals to a computer. The sensing signals are analyzed by the program of the data acquisition system. The data are then displayed on the display panel.

### BACKGROUND OF THE INVENTION

1. Field of the Invention

The present invention relates to solid-state urea biosensors and an accompanying data acquisition system and more particularly to a solid-state urea biosensor having a pH-sensitive membrane made of SnO₂.

2. Description of the Prior Art

Potentiometric biosensors have been widely used in biotechnologies, medical assays, and environmental protections. Typical conventional potentiometric biosensors all require a commercial reference electrode to provide a reference potential to analyte solutions (W. Torbicz, D. G. Pijanowska, 1997, “pH-ISFET based urea biosensor”, Sensors and Actuators B, 44: 370-376) relative to the detected signals. The prices for the commercial reference electrodes are high. The commercial reference electrodes are made of glass, which confines applications on miniaturized inspection and cuts down on fabrication costs, and as a consequence it is difficult for such electrodes to be fabricated into biosensors for disposable use.

There are quite many researches to make solid-state biosensors by exploiting various technologies, for instance, solid-state reference electrode technique (L. Bousse, J. Shott, and J. D. Meindl, 1988, “A process for the combined fabrication of ion sensors and CMOS circuits”, IEEE Electron Device Letters, 9: 44-46; I. Y. Huang and R. S. Huang, 2002, “Fabrication and characterization of a new planar solid-state reference electrode for ISFET sensors”, Thin Solid Films, 406: 255-261), and differential pair technique (H. S. Wong and M. H. White, “A self-contained CMOS integrated pH sensor”, Proceedings of the 1988 International Electron Devices Meeting, San Francisco, Calif., 11-14 Dec., 1988, pp. 658-661; H. S. Wong, and M. H. White, 1989, “A CMOS-integrated ‘ISFET-operational amplifier’ chemical sensor employing differential sensing”, IEEE Transactions on Electron Devices, 36: 479-487) are under study respectively in order to overcome the drawbacks of the conventional reference electrodes in the fabrication of solid-state typed sensors. Among them, the solid-state reference electrode technique seems to have characteristics similar to those of the commercial reference electrodes, but it's difficult in the fabrication to stabilize its characteristics. The differential pair technique makes use of a quasi-reference electrode for providing the readout circuit and analyte solution a standard potential, where the quasi-reference electrode is made of electrically conductive material which simplifies the production. Moreover, since the output signal is a potential difference between two sensing devices, which cancels out non-ideal effects of the sensing devices, such as drift phenomena, the differential pair technique is superior in overcoming technical obstacles borne in the conventional reference electrodes, making it advantageous in commercialization. Articles on fabricating biosensors by this technique have been found in quite many journals (W. Sant, M. L. Pourciel, J. Launay, T. Do Conto, A. Martinez, P. Temple-Boyer, 2003, “Development of chemical field effect transistors for the detection of urea”, Sensors and Actuators B, 95: 309-314; B. Pala´n, F. V. Santos, J. M. Karam, B. Courtois, M. Husa´k, 1999, “New ISFET sensor interface circuit for biomedical applications”, Sensors and Actuators B, 57: 63-68). It is apparent that the differential pair technique has been technically mature to be useful and reliable; however, although the technique is successful in the development, but conventional types demand more than two materials for forming the quasi-reference electrode and a reference electrode, which complicates the whole fabrication procedure and raises its costs.

The concentration of an analyte is generally calculated by the use of a linear regression method for the conventional biosensor inspections, where the available linear region affects the limits of inspection of the biosensors. Moreover, the quality of the readout system in the backend also affects signal acquisition and analysis of the biosensors, hence it is essential for the readout system to have a considerable degree of accuracy.

U.S. Pat. No. 5,309,085 issued May 1994 to Byung Ki Sohn and entitled “Measuring circuit with a biosensor utilizing ion sensitive field effect transistors (ISFET)” discloses a measuring circuit with a biosensor utilizing ion sensitive field effect transistors (FET) having a simplified structure as its basis. The measuring circuit comprises two ISFETs, an enzyme FET having an enzyme sensitive membrane on its gate and a reference FET, and a differential amplifier for amplifying the outputs of the enzyme FET and the reference FET. The drift phenomena of the ISFETs due to the use of a non-stable quasi-reference electrode as well as the temperature dependence thereof can be eliminated by the differential amplifier consisting of MOSFETs having the same channel as the ISFETs. The patent is advantageous in the integration of the ISFET biosensor and the measuring circuit into one single chip, the elimination of the drift phenomena of the biosensor, and the fabrication into a monolithic device, whereas the disadvantage is that under the integration of the measuring circuit and the biosensor, the yield rate for fabricating ISFETs alone will affect the yield rate of the whole system. Besides, while semiconductor processes approach miniaturization, the sensors fail to follow suit only for stability's sake; this situation prevents innovative semiconductor processes from being adopted for fabricating sensors, which in turn raises the costs and deters the commercialization.

U.S. Pat. No. 5,858,186 issued January 1990 to Robert S. Glass and entitled “Urea biosensor for hemodialysis monitoring” discloses an electrochemical sensor capable of applying in the hemodialysis monitoring, which is fabricated for the measurement of the pH change produced in an aqueous environment by the products of enzyme-catalyzed hydrolysis of urea. The concentration of urea is estimated through the pH change measured by for instance a potentiometric pH sensor. Due to a low fabrication cost, the potentiometric pH sensors have advantages in suiting mass fabrication and the potential for disposable use. In addition, the sensor could also be used in at-home hemodialysis monitoring. The use of the potentiometric pH sensor requires it to be combined with a stable reference electrode. The cost of the reference electrode also influences the fabrication costs of the sensors. The patent offers no effective technique to lessen the influence by the reference electrode or to lower the fabrication costs. A selection of a suitable reference electrode somehow could contribute to its practical use.

U.S. Pat. No. 5,945,343 issued August 1999 to Christiane Munkholm and entitled “Fluorescent polymeric sensor for the detection of urea” discloses a fluorescent polymeric sensor capable of detecting urea. The urea sensor is configured in a tri-level structure: the top layer is made with a protonated pH sensitive fluorophore immobilized in a hydrophobic polymer; the second layer comprises a polymer and urease; and the third layer a polymer. The sensor disclosed by this patent has a plain structure that enables the fabrication of miniaturized sensor and allows for disposable use. Unfortunately, the patent fails to make any improvements on the stability of the operation and fabrication of the optical inspection system made with optical sensing devices. Therefore, viewing from a loaded standpoint on sensors, the optical sensor system has higher fabrication costs than those of potentiometric and amperometric sensor systems, which is its major drawback.

U.S. Pat. No. 5,922,183 issued July 1999 to Rauh; R. David and entitled “Metal oxide matrix biosensors” discloses a thin film matrix for biomolecules, suitable for forming electrochemical biosensors comprising a general class of materials known as hydrous metal oxides which are also conductive or semiconductive of electrons and which have been shown to have excellent stability against dissolution or irreversible reaction in aqueous and nonaqueous solutions. The thin film composites of the oxides and biological molecules such as enzymes, antibodies, antigens and DNA strands can be used for both amperometric and potentiometric sensing. Hydrous IrO₂ is the preferred matrix embodiment, but conducting or semiconducting oxides of Ru, Pd, Pt, Zr, Ti and Rh thereof have similar features. The hydrous oxides are very stable against oxidation damage. The patent discloses the fabrication of thin film matrix for biomolecules, but overlooks the fabrication of reference electrode and read-out system. The patent hasn't actually resolved the influences by the reference electrode and read-out system yet.

U.S. Pat. No. 4,879,517 issued November 1989 to Connery, et al. and entitled “Temperature compensation for potentiometrically operated ISFETS” discloses compensation for the temperature sensitivity of the output of a potentiometrically operated ISFET probe whose drain-source voltage and drain-source current are held constant is provided by using a Nernstian temperature correction of the difference between the ISFET output and the isopotential voltage of the probe and offsetting the resulting difference by the isopotential plon value. An ISFET/NISFET pair provides a cancellation of variations due to manufacturing. The patent succeeds in providing temperature compensation for measurements made with potentiometrically operated ion-sensitive field effect transistor (ISFET), but ignores the increase in production costs.

U.S. Pat. No. 4,691,167 issued September 1987 to v. d. Vlekkert, et al. and entitled “Apparatus for determining the activity of an ion (plon) in a liquid” discloses an apparatus for determining the plon in a liquid which comprises a measuring circuit including an ion sensitive field effect transistor (ISFET), a reference electrode, a temperature sensor, amplifiers and control, computing and memory circuits operable to maintain two of the following three parameters: V_{G S}, V_{D S} and I_{D} at a constant value so that the third parameter can be used for determining the plon. The plon sensitivity of the apparatus as a function of temperature or the variation of the I_{D} as a function of the temperature are controlled by controlling the V_{G S} so that the plon can be calculated from a formula stored in the memory. The patent is advantageous in providing temperature compensation by maintaining two of the three parameters for controlling the operation of the ISFET at a constant value, and the third parameter is used for determining the ion activity; however, concerns over its operation and cost make the apparatus an expensive one.

U.S. Pat. No. 5,602,467 issued February 1997 to Krauss, et al. and entitled “Circuit for measuring ion concentrations in solutions” discloses a circuit layout for measuring ion concentrations in solutions using ion sensitive field effect transistors. The circuit layout makes it possible to represent the threshold voltage difference of two ISFETs directly and independently of technological tolerances, operationally caused parameter fluctuations, and ambient influences. The circuit layout includes two measuring or test amplifiers, with in each case two differently or identically sensitive ISFETs and two identical FETs. The ISFETs and FETs are connected in such a manner that the output of the first measuring amplifier occurs the difference of the mean value of the two ISFET threshold voltages and the FET threshold voltage, and the output of the second measuring amplifier occurs the difference of the two ISFET threshold voltages. The output of the first amplifier is connected to the common reference electrode of the four ISFETs. The measurement by the circuit layout of the patent is advantageously independent of technologically caused tolerances of components, age-caused threshold voltage drifts, or fluctuations to operating parameters, such as temperature or operating voltage changes. On the contrary, it implies higher complexities in technical and operational aspects and hardly for disposable use.

It is amply evident from the above that the biosensors and their accompanying signal acquisition systems as practiced before the present invention have either failed in commercialization or required cumbersome technologies, and are really not sound designs for the practical usage. There is still considerable room for improvement.

### SUMMARY OF THE INVENTION

It is an object of the present invention to provide a solid-state urea biosensor suitable for disposable use by applying differential pair technique in the fabrication of semiconductor potentiometric sensors.

It is another object of the present invention to provide a data acquisition system for the solid-state urea biosensors, which is a data acquisition system that can be joined together with a differential pair biosensor. The integration of the method with the solid-state urea biosensor of the present invention is capable of accomplishing a complete cycle of measurement and analysis, and ready for fabrication. A further goal of plain process, low cost and accuracy could very possibly be reached.

In order to achieve the above object, the solid-state urea biosensor and its data acquisition system according to the present invention comprises:

A solid-state urea biosensor comprises a substrate, on top of the substrate there are three sensing areas locating in an electrical insulating layer and separated by the insulating material, where there is no electrical conduction among each area. A conductive layer is fixed on the substrate of each sensing area, and a pH sensitive membrane on top of each conductive layer, where an enzyme sensitive membrane is laid on the pH sensitive membrane of one of the sensing area, to form an enzyme working electrode while a quasi-reference electrode and a reference electrode are formed for the other two sensing areas respectively. A conducting wire is set for each sensing area for the transmission of sensed signals of each sensing area;

A data acquisition system for solid-state urea biosensors, employs an instrumentation amplifier to acquire measured signal of an analyte by the solid-state urea biosensor, sends the signal to a low-pass filter for the attenuation of high-frequency noises, relays the noise-free signal through a data acquisition card to a computer, displays the digitized signal on a readout potential display panel of the computer, instructs a signal analysis program to analyze the digitized signal, where the signal analysis program comprises an analysis function and a parameter setting panel, for real-time calibration of arithmetic parameters and calculation of concentration of the analyte, and shows the calculated outcome on an analyte concentration display panel;

wherein the substrate is an insulating substrate (e.g. glass) or a non-insulating substrate (e.g. indium tin oxide glass or tin dioxide glass, SnO₂);

wherein the electrical insulating layer is biphenol epoxy (BP);

wherein the conductive layer is indium tin oxide or aluminum;

wherein the pH sensitive membrane is SnO₂;

wherein the enzyme is urease;

wherein the enzyme membrane immobilize the enzyme by physical entrapment or covalent bonding;

wherein the physical entrapment employs a polymer (e.g. polyvinyl alcohol bearing styrylpyridium groups, PVA-SbQ) to immobilize the enzyme;

wherein the covalent bonding employs a chemical substance (e.g. 3-glycidoxypropyltri-methoxysilane, GPTS) to immobilize the enzyme;

wherein the quasi-reference electrode is a SnO₂ membrane capable of providing a standard potential in between the circuit and the solution;

wherein the enzyme working electrode is an enzyme sensitive membrane used for reacting with analyte solution to provide reaction potential;

wherein the reference electrode is a SnO₂ membrane capable of providing the enzyme working electrode with a reference potential;

wherein the instrumentation amplifier comprises operational amplifiers, resistors and a variable resistor, where the variable resistor is used for gain changing, and through the use of the instrumentation amplifier, a common mode rejected signal is effectively acquired and being amplified to get a gained output signal;

wherein the low-pass filter comprises operational amplifiers, resistors, capacitors and a variable resistor, and the low-pass is targeted as removing high-frequency noises and probably for amplifying the signal, where the variable resistor is used for balancing the phase shift;

wherein the data acquisition card is a GPIB card or DAQ card, capable of converting a signal from an analog form to a digital form, and sending the digital signal to a computer;

wherein the readout potential display panel is used for changing signal unit and interval, altering time interval for acquiring signal, and real-time displaying the reaction potential of the solid-state urea biosensor to analyze if the variation of the reaction potential is right;

wherein the signal analysis program can be LabVIEW or HP VEE, which manipulates the digital signal into analysis, arithmetic and storage;

wherein the analysis function is a sigmoid regression function, capable of calculating a measured signal of an analyte by the solid-state urea biosensor into a concentration of that analyte, and the sigmoid regression function takes the mean value of the measured signal for analysis, and determines the parameter values;

wherein the parameter setting panel is used to set: the parameters of an arithmetic function, program executed switch, channel selection of data acquisition card, location of the acquired data in storage, and time interval of acquisition. The parameters of the arithmetic function can be set through the use of either the linear regression function or the sigmoid regression function. The program executed switch issues an interruption on program execution. The channel selection of data acquisition card decides a routing choice for acquiring signal, which is to avoid job quitting for single-channel collapse. The location of the acquired data in storage can be in a computer hard disk, a flash disk, a portable hard disk, or a network hard disk. The time interval of acquisition is dependent on the reaction time of the solid-state urea biosensor;

wherein the analyte concentration display panel shows output potential, concentration of urea and warning lamps. The output potential is the actual potential outputted by the solid-state urea biosensor. The unit for a concentration of urea can be either mg/100 ml or molarity for avoiding troublesome unit conversion. The warning lamps comprise a too-high lamp, a normal lamp, and a too-low lamp, which is used for signaling the operator a comparison between the analyte and clinical values. As one lamp lightens, it means that a sensed concentration is located in the too-high range, normal range, or too-low range. As two lamps lighten, it means that a sensed concentration is located in between the too-high range and the normal range, or in between the normal range and the too-low range.

These features and advantages of the present invention will be described in detail with reference to the accompanying drawings.

### BRIEF DESCRIPTION OF THE DRAWINGS

FIG. 1 is an overview illustrating the processes of fabricating the solid-state urea biosensor and its data acquisition system according to the present invention;

FIG. 2 shows a schematic plan view of the solid-state urea biosensor;

FIG. 3 shows a cross-sectional view of the solid-state urea biosensor;

FIG. 4 shows a graphic representation of a measurement by the solid-state urea biosensor;

FIG. 5 shows the readout circuit diagram of the signal acquisition system;

FIG. 6 shows a full view of the front panel of the signal acquisition system;

FIG. 7 shows a full view of the analysis program of the signal acquisition system;

FIG. 8 shows the variation over time of the concentration of the analytic solution with respect to the output potential of the solid-state urea biosensor;

FIG. 9 shows a contrast between the mean sensed signal and linear regression values; and

FIG. 10 shows a contrast between the mean sensed signal and sigmoid regression values.

### DETAILED DESCRIPTION OF THE PREFERRED EMBODIMENT

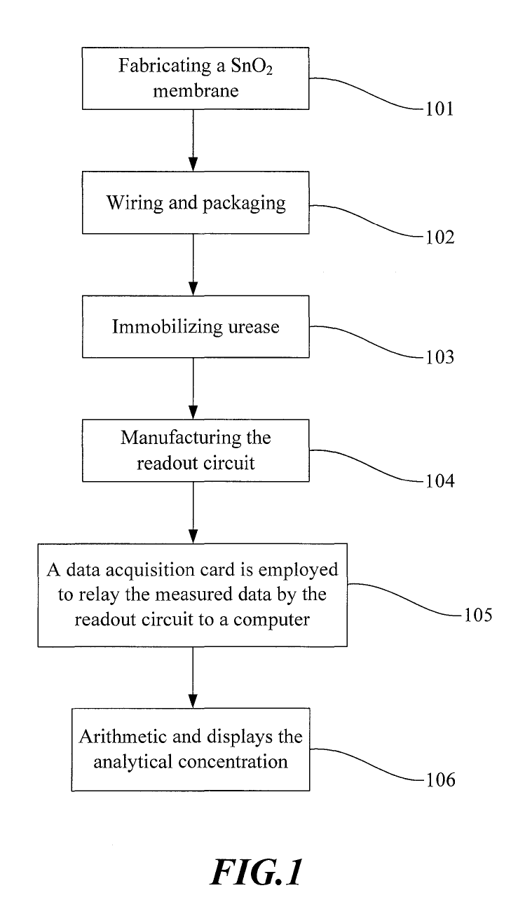

A series of major processes for fabricating the solid-state urea biosensor and its accompanying data acquisition system according to the present invention is shown in FIG. 1. Referring to FIG. 1, the entire process starts with the fabrication of a SnO₂ membrane 101 which is for the production of a pH sensor and a quasi-electrode, followed by wiring and packaging 102 for achieving a basic structure of a solid-state sensor, then immobilization of urease 103 on the SnO₂ membrane 101 to add on bio-sensing features, thus completing the fabrication of the solid-state urea biosensor. The readout circuit 104 is then manufactured for gathering the measured signals obtained by the solid-state urea biosensor. A data acquisition card is employed to relay the measured data by the readout circuit 104 to a computer 105. Finally, the signal analysis program used by the present invention manipulates the arithmetic and displays the analyte concentration 106. The process of making the solid-state urea biosensor and its accompanying signal acquisition system is then accomplished.

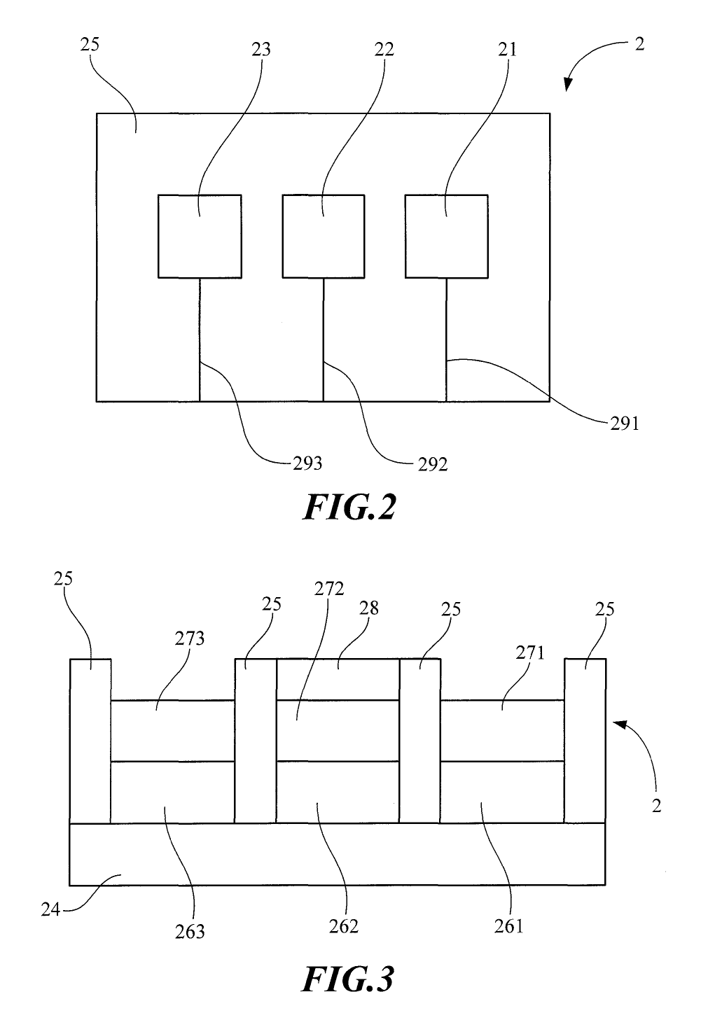

A schematic plan view of the solid-state urea biosensor according to the present invention is shown in FIG. 2. FIG. 3 shows a cross-sectional view. Referring to FIG. 3, the solid-state urea biosensor has a substrate 24 made of an insulating substance, e.g. glass. The substance used for the substrate 24 could also be a non-insulating substance: indium tin oxide (ITO) or tin dioxide (SnO₂). On the top of the substrate 24 there are three sensing areas located in an electrical insulating layer 25 and separated by the insulating material made of biphenyl epoxy (BP). The three sensing areas do not conduct electricity to each other. Three conductive layers: 261, 262, and 263 are fixed on the substrate within each sensing area. The three conductive layers, 261, 262, and 263, are made of indium tin oxide (ITO) as buffer layers. Otherwise, aluminum could also be used as the substance for the three conductive layers, 261, 262, and 263. A layer of SnO₂ thin film is sputtered on top of each conductive layers, 261, 262, and 263 as a pH sensitive membrane: 271, 272, and 273. An enzyme sensitive membrane 28 is laid on the pH sensitive membrane 272 to form an enzyme working electrode 22, where the enzyme is urease. The enzyme is immobilized on the enzyme sensitive membrane 28 by physical entrapment or covalent bonding. The physical entrapment is a method that employs a polymer, polyvinyl alcohol bearing styrylpyridium groups (PVA-SbQ), to immobilize enzyme. The covalent bonding is a method that employs a chemical substance, 3-glycidoxypropyltri-methoxysilane (GPTS), to immobilize the enzyme. A quasi-reference electrode 23 and a reference electrode 21 are formed in the other two sensing areas, respectively. Each sensing area has a conducting wire: 291, 292, and 293, respectively, for transmitting its sensed signals.

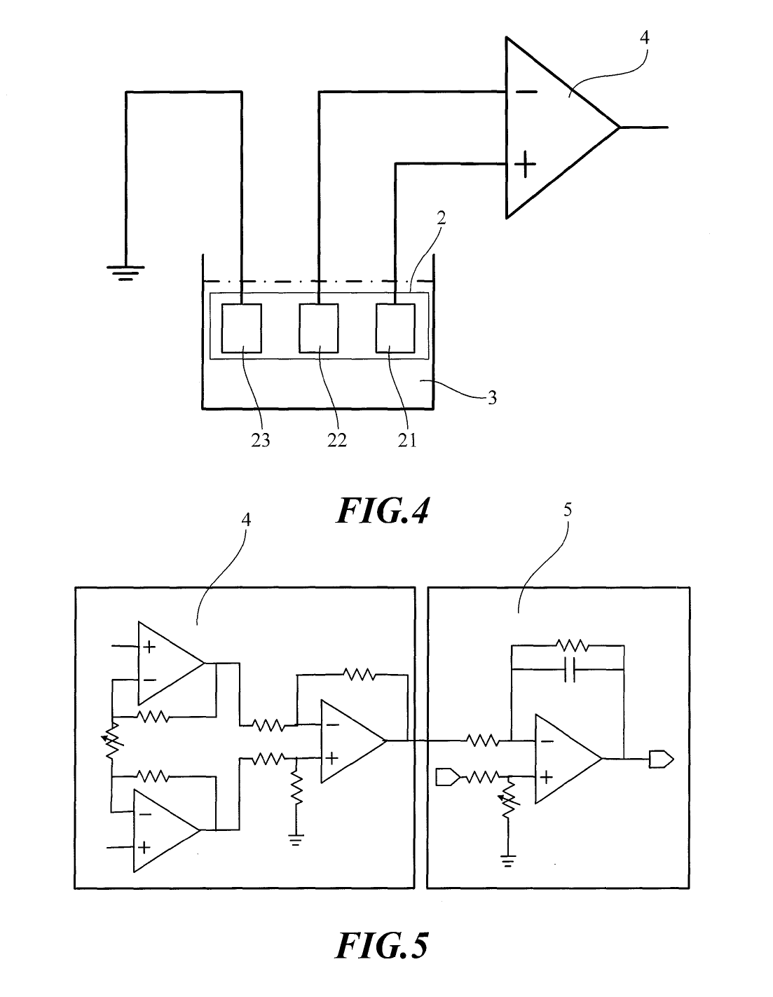

FIG. 4 shows a graphic representation of a measurement by the solid-state urea biosensor according to the present invention. Referring to FIG. 4, the solid-state urea biosensor 2 is immersed in the analyte solution 3. The quasi-reference electrode 23 is grounded to define the basic electric potential of the analyte solution 3, and provides the circuit the same ground voltage to stabilize the signal of the biosensor. The reference electrode 21 is connected to the plus input terminal of the instrumentation amplifier 4, supplying a reference voltage which is an integral part of a differential signal. The reference voltage also determines a reference potential of the analyte solution 3 where the solid-state urea biosensor 2 is soaked in. The enzyme working electrode 22 is connected to the minus input terminal of the instrumentation amplifier 4, supplying a signal, which determines the working potential of the solid-state urea biosensor. The differential signal to the input terminals of the instrumentation amplifier 4 is the potential difference between the reference electrode 21 and the enzyme working electrode 22. The enzyme working electrode 22 is immobilized on the pH sensitive membrane 272 made of a SnO₂ membrane. The reference electrode 21 also comprises a SnO₂ membrane. As long as both the enzyme working electrode 22 and the reference electrode 21 are soaked in the same analyte solution 3, they have the same ground potential under measurement. The differential signal is the actual sensed signal by the enzyme working electrode 22. Therefore, the solid-state urea biosensor 2 according to the present invention is able to effectively eliminate the drift phenomena due to the quasi-reference electrode and the temperature dependence thereof, which helps raise the accuracy of the sensor.

FIG. 5 shows the readout circuit diagram of the signal acquisition system for a solid-state urea biosensor according to the present invention. Referring to FIG. 5, the readout circuit comprises the instrumentation amplifier 4 and a low-pass filter 5. The instrumentation amplifier 4 amplifies the differential signal at its input terminals, which are connected to the enzyme working electrode 22 and the reference electrode 21, respectively. The instrumentation amplifier 4 comprises operational amplifiers, resistors and a variable resistor, where the variable resistor is used for gain changing, and through the use of the instrumentation amplifier 4, a common mode rejected signal is effectively acquired and amplified to get a gained output signal. The usage of an amplifier for medical applications such as the present invention has to have high input impedances, very high common-mode rejection, high gain, and low noise, so the instrumentation amplifier 4 is the right choice and operable to cancel out the noise at its input terminals. The low-pass filter 5 comprises operational amplifiers, resistors, capacitors and a variable resistor for attenuating high-frequency noises while passing those of lower frequency unchanged. A variable resistor can be used for adjusting the phase shift. The low-pass filter 5 can not only attenuate unwanted high-frequency noise but also eliminate false lower frequency signal and interference introduced prior to sampling when processing signal with a data acquisition system.

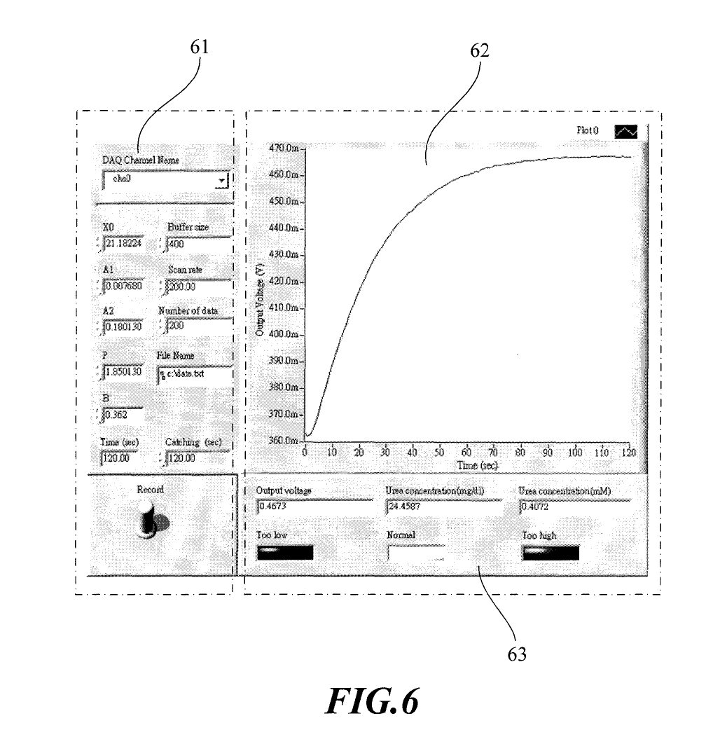

The signal sensed by the sensor is read to the readout circuit and sent to a computer through a data acquisition card. The data acquisition card can be a GPIB card or DAQ card, capable of converting an analog signal into a digital signal and then relaying it to a computer. The concentration of the analyte can then be calculated by a signal analysis program used by the present invention. The signal analysis program analyzes, calculates, and stores the digital signal. The signal analysis program can be LabVIEW or HP VEE. The display panels shown in FIG. 6 offered by the signal analysis program are: a parameter setting panel 61, a readout potential display panel 62, and an analyte concentration display panel 63.

Referring to FIG. 6, the parameter setting panel 61 is used to set: selected channel of data acquisition, parameters of arithmetic function, file name and storage location of acquired data, time interval for signal acquisition. The parameter setting panel 61 can modify the required parameters as any changes occur to the sensor. It's a user-friendly design and handy in use. The readout potential display panel 62 is used for displaying the variation of the reaction potential of the sensor, and for changing the signal unit and interval. It could also be used for changing the time interval for acquisition, and for real-time display of the reaction potential of the sensor. The panel 62 is used to analyze if the variation of the reaction potential is right. The potential value shown on the panel 62 is the actual sensed signal representation of the concentration of the desired substrate. The analyte concentration display panel 63 displays an output potential, a concentration of analyte (i.e. concentration of urea) and warning lamps; the appropriate warning lamps lighten according to whether the concentration of analyte lies in the normal range of clinical values or outside the range. The unit for the concentration of urea can be mg/100 ml or molarity, which is for avoiding troublesome unit conversion. The warning lamps comprise a too-high lamp, a normal lamp, and a too-low lamp, signaling to the operator a comparative relationship between the analyte and clinical values. As one lamp lightens, it means that an analyte concentration lies in the corresponding range: too-high, normal, or too-low, respectively. As two lamps lighten, it means that an analyte concentration lies in between the too-high range and the normal range, or between the normal range and the too-low range. The too-high lamp lightening means that the concentration of urea is higher than 39 mg/dl, while the normal lamp lightening means that the concentration lies between 15-40 mg/dl, and the too-low lamp lightening means that the concentration is lower than 16 mg/dl.

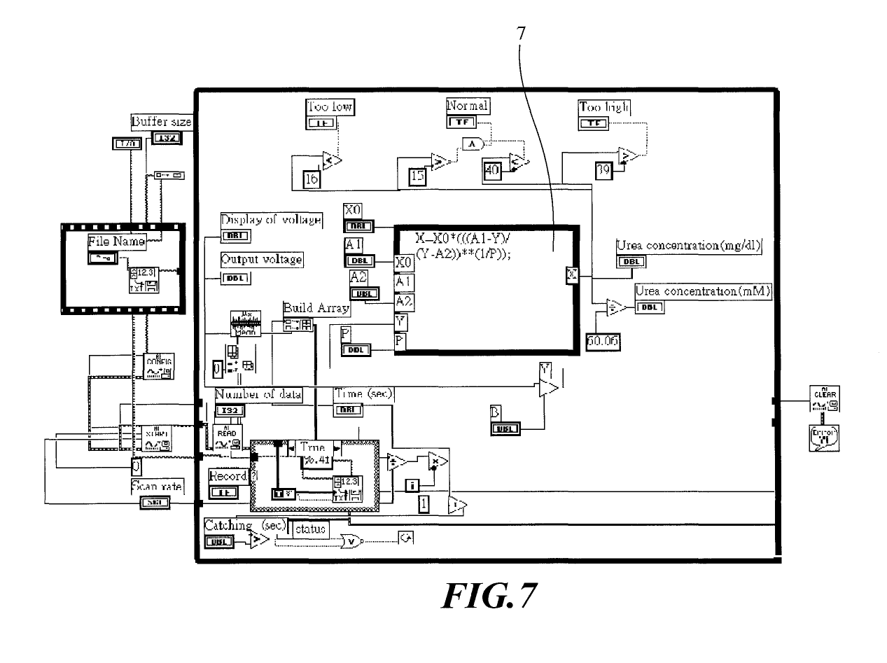

FIG. 7 shows a full view of the contents of the analysis program. Referring to FIG. 7, the analysis function 7 is used for calculating a concentration of urea by the sigmoid regression method, suitable for analyzing biosensor signal and capable of raising its accuracy. The analyzed data can be stored in a computer, in a computer hard disk, flash disk, portable hard disk, or network hard disk for a long term case history tracing and analysis.

### EXAMPLE 1

Signal Acquisition and Analysis for the Solid-State Urea Biosensor

The present embodiment utilizes the solid-state urea biosensor 2 according to the present invention (referring to FIG. 2). The measurement architecture is shown in FIG. 4, wherein the solid-state urea biosensor 2 is soaked in the analyte solution 3, the quasi-reference electrode 23 is grounded, the reference electrode 21 is connected to the plus input terminal of the instrumentation amplifier 4, and the enzyme working electrode 22 is connected to the minus input terminal of the instrumentation amplifier 4. The solid-state urea biosensor produces a differential signal between the two input terminals of the instrumentation amplifier 4. By setting a gain of 1, the signal level at the output terminal of the instrumentation amplifier 4 is equal to its input differential value. The noise in the circuit is filtered by a low-pass filter 5 (referring to FIG. 5). The data acquisition card is used to relay the noise-free signal to the computer and show the digitized signal on the readout potential display panel 62 (referring to FIG. 6). The panel 62 monitors whether the output signal of the solid-state urea biosensor 2 is in a proper potential range, and determines the ground potential. The arithmetic parameter setting panel 61 is used to assign values to the parameters of the arithmetic function (referring to FIG. 7). The arithmetic function takes care of the calculation to obtain the concentration of analyte solution 3, which is then displayed on the analyte concentration display panel 63. By comparing the concentration of the analyte solution 3 with the clinical values, whether the concentration is normal, too-high or too-low, it will be shown on the panel 63 to get user's attention. The concentration data is stored in the computer with a filename “data.txt” for long term case history tracing and analysis.

### EXAMPLE 2

Solid-State Urea Biosensor Output Potential Variation

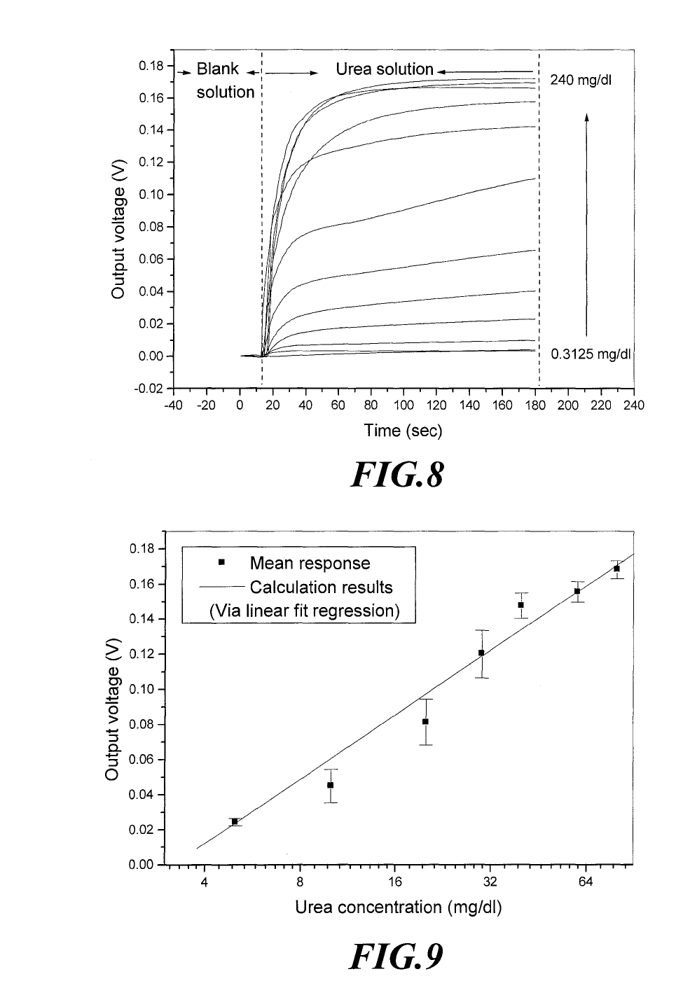

Place the solid-state urea biosensor 2 (referring to FIG. 2) according to the present invention in a separate analyte solution 3 (referring to FIG. 4). With a measurement method same as in the embodiment 1, the setting according to the present embodiment includes: grounding the quasi-reference electrode 23, connecting the reference electrode 21 to the plus input terminal of the instrumentation amplifier 4, connecting the enzyme working electrode 22 to the minus input terminal of the instrumentation amplifier 4, producing a differential signal outputted by the solid-state urea biosensor at the input terminals of the instrumentation amplifier 4, setting gain to 1, filtering the noise in the circuit by the low-pass filter 5 (referring to FIG. 5), sending the noise-free signal to a computer by means of the data acquisition card, storing the signal in a hard disk with a filename “data.txt”, and finally employing a software to manipulate the time variation of the concentration of the analyte solution 3 with respect to the output potential and depict the result in FIG. 8. Referring to FIG. 8, the response time of the solid-state urea biosensor 2 according to the present invention varies, depending on the concentration of the analyte solution, approximately 60-120 seconds, with a measurement range of urea concentration between 0.3125-240 mg/dl, which apparently covers the clinical normal range, 15-40 mg/dl. The solid-state urea biosensor 2 according to the present invention has a maximum output potential of 175 mV which is proportional to the urea concentration. All the measured data are stored for later analysis.

### EXAMPLE 3

Analysis of Solid-State Urea Biosensor Characteristics by the Linear Regression

The present embodiment analyzes the traits of the solid-state urea biosensor by the linear regression technique. The analyzed outcome is shown in FIG. 9, wherein the linear area for the solid-state urea biosensor falls in the concentrations 5-80 mg/dl which covers the clinical normal range, 15-40 mg/dl. The deviation between the mean sensed signal and the linear regression value gets greater in the concentrations around 10 mg/dl up to 20 mg/dl. This means the linear regression is a viable but not preferred technique for the analysis of solid-state urea biosensor characteristics.

### EXAMPLE 4

Analysis of Solid-State Urea Biosensor Characteristics by the Sigmoid Regression

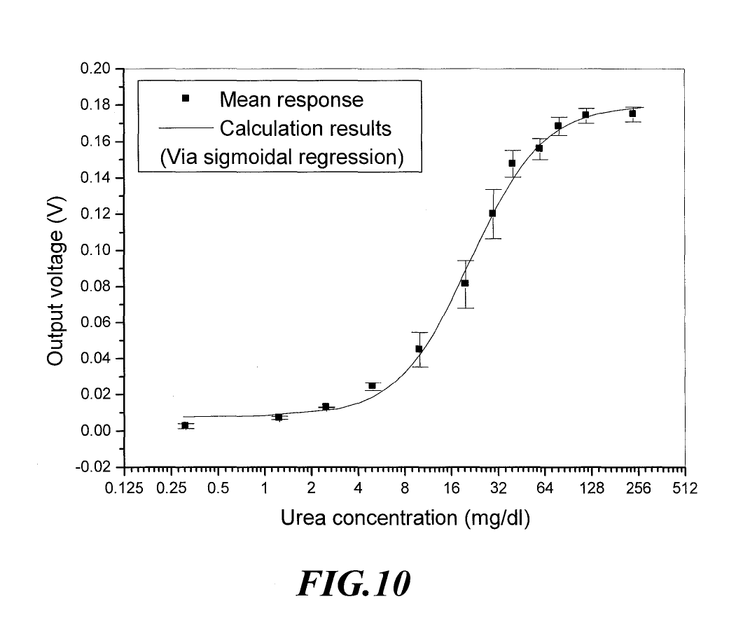

The present embodiment analyzes the traits of the solid-state urea biosensor by the sigmoid regression technique. The analyzed outcome is shown in FIG. 10, wherein the range available for the calculation for the solid-state urea biosensor falls in the concentrations 0.3125-240 mg/dl which covers the clinical normal range, 15-40 mg/dl. Within the range, the calculated values approach the measurements, and the deviation between the mean sensed signal and the linear regression value is smaller. Therefore, the sigmoid regression is the preferred choice for the arithmetic function of the signal analysis program of the present invention. The concentration of analyte solution can be measured to a sizable degree of accuracy and within a broad scope by the sigmoid regression.

The solid-state urea biosensor and its data acquisition system according to the present invention, when compared with the prior art quoted in the foregoing description, has the following advantages:

1. The solid-state urea biosensor of the present invention employs a conductive substance as the sensing material. The quasi-electrode and the reference electrode are made of a single material, offering the circuit and the solution a stable reference potential. The present invention uses only a single substance for fabricating both electrodes, considerably simplifying the fabrication process and and minimizing the costs, which are conducive to mass production of disposable biosensors.

2. The solid-state urea biosensor of the present invention employs a single material to fabricate the quasi-electrode and the reference electrode, thus effectively eliminating the drift phenomena due to the quasi-reference electrode and the temperature dependence thereof and therefore helping to raise the accuracy of the sensor.

3. The data acquisition system accompanied with the solid-state urea biosensor offered by the present invention is the kind of data acquisition system capable of integrating with the differential biosensor. The method favors effective acquisition of signals of the sensor, and employs the sigmoid regression technique for the calculation of analyte solution. In contrast to the prior linear analysis, the method used by the present invention is more stable, capable of widening the scope of concentration calculation and raising the accuracy.

4. The data acquisition system accompanied with the solid-state urea biosensor offered by the present invention features handy software operation panels that ease user operations and provide advantages of real time function: adjustment, display, calculation, and analysis, etc.

5. The data acquisition system accompanied with the solid-state urea biosensor offered by the present invention is applicable to at-home medical inspection through real-time measurement and analysis software for handling the measured data. Moreover, the outcome of the inspection could be sent to a hospital or clinic for building a case history.

While the present invention has been illustrated in detail herein with reference to the preferred embodiments thereof, the present invention is not intended to be limited by the embodiments. Any equivalent embodiments or modifications without departing from the spirit and scope of the present invention, for instance, equivalent embodiments of variations, of the substrate of the solid-state urea biosensor, or of the material used by the electrodes are therefore intended to be embraced.

From the above description, the present invention provides not only a novel method of fabricating sensor, signal acquisition, arithmetic approach, but a useful improvement thereof over the prior biosensors.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A solid-state urea biosensor comprising: a substrate, an insulating layer thereon, and three sensing areas surrounded and separated from each other by said insulating layer, wherein each of said three sensing areas comprises a conductive layer fixed on said substrate and a pH sensitive membrane laid on top of said conductive layer; one of said three sensing areas further comprises an enzyme sensitive membrane laid on said pH sensitive membrane to form an enzyme working electrode; the other two of said three sensing areas form a quasi-reference electrode and a reference electrode, respectively; and a conducting wire is set for each of said three sensing areas for transmission of sensed signals.

2. A solid-state urea biosensor according to claim 1, wherein the substrate is glass.

3. A solid-state urea biosensor according to claim 1, wherein the substrate is indium tin oxide (ITO) glass or tin dioxide (SnO₂) glass.

4. A solid-state urea biosensor according to claim 1, wherein the insulating layer is biphenol epoxy (BP).

5. A solid-state urea biosensor according to claim 1, wherein the conductive layer is ITO_or aluminum.

6. A solid-state urea biosensor according to claim 1, wherein the pH sensitive membrane is SnO₂.

7. A solid-state urea biosensor according to claim 1, wherein an enzyme is immobilized on the enzyme sensitive membrane by physical entrapment or covalent bonding.

8. A solid-state urea biosensor according to claim 7, wherein the enzyme is urease.

9. A solid-state urea biosensor according to claim 7, wherein the physical entrapment employs a polymer to immobilize the enzyme.

10. A solid-state urea biosensor according to claim 9, wherein the polymer is a polyvinyl alcohol bearing styrylpyridium groups (PVA-SbQ).

11. A solid-state urea biosensor according to claim 7, wherein the covalent bonding employs a chemical substance to immobilize the enzyme.

12. A solid-state urea biosensor according to claim 11, wherein the chemical substance is 3-glycidoxypropyltrimethoxysilane (GPTS).

13. A solid-state urea biosensor according to claim 1, wherein the conductive layer of the quasi-reference electrode is a SnO₂ membrane.

14. A solid-state urea biosensor according to claim 1, wherein the enzyme working electrode is used for reacting with an analyte solution to provide a reaction potential.

15. A solid-state urea biosensor according to claim 1, wherein the conductive layer of the reference electrode is a SnO₂ membrane, and the reference electrode provides the enzyme working electrode with a reference potential.

16. A data acquisition system for a solid-state urea biosensor, employing an instrumentation amplifier to amplify measured signal of an analyte acquired by the solid-state urea biosensor, sending the signal to a low-pass filter for the attenuation of high-frequency noises, relaying the noise-free signal through a data acquisition card to a computer, displaying the digitized signal on a readout potential display panel of the computer, instructing a signal analysis program to analyze the digitized signal, where the signal analysis program comprises an analysis function and a parameter setting panel, for real-time calibration of arithmetic parameters and calculation of concentration of the analyte, and showing the calculated outcome on an analyte concentration display panel.

17. A data acquisition system for a solid-state urea biosensor according to claim 16, wherein the solid-state urea biosensor is the solid-state urea biosensors according to claim 1.

18. A data acquisition system for a solid-state urea biosensor according to claim 16, wherein the instrumentation amplifier comprises a plurality of operational amplifiers, a plurality of resistors and a variable resistor, wherein the variable resistor is used for gain changing, and through the use of the instrumentation amplifier, a common mode rejected signal is effectively acquired and amplified to get a gained output signal.

19. A data acquisition system for a solid-state urea biosensor according to claim 16, wherein the low-pass filter comprises a plurality of operational amplifiers, a plurality of resistors, a plurality of capacitors, wherein the variable resistor is used for balancing the phase shift.

20. A data acquisition system for a solid-state urea biosensor according to claim 16, wherein the data acquisition card is a GPIB card or DAQ card, capable of converting a signal from an analog form to a digital form and sending the digital signal to a computer.

21. A data acquisition system for a solid-state urea biosensor according to claim 16, wherein the readout potential display panel is used for changing signal unit and interval, altering time interval for acquiring signal, and real-time display of the reaction potential of the solid-state urea biosensor to analyze if the variation of the reaction potential is right.

22. A data acquisition system for a solid-state urea biosensor according to claim 16, wherein the analysis function is a sigmoid regression function, capable of calculating a measured signal of an analyte by the solid-state urea biosensor into a concentration of that analyte.

23. A data acquisition system for a solid-state urea biosensor according to claim 22, wherein the sigmoid regression function takes the mean value of the measured signal for analysis, and determines the parameter values.

24. A data acquisition system for a solid-state urea biosensor according to claim 16, wherein the parameter setting panel is used to set the parameters of an arithmetic function, program executed switch, channel selection of the data acquisition card, location of the acquired data in storage, and time interval of acquisition.

25. A data acquisition system for a solid-state urea biosensor according to claim 24, wherein the parameters of the arithmetic function are set through the use of either the linear regression function or the sigmoid regression function.

26. A data acquisition system for a solid-state urea biosensor according to claim 24, wherein the program executed switch issues an interruption on program execution.

27. A data acquisition system for a solid-state urea biosensor according to claim 24, wherein the channel selection of the data acquisition card decides a routing choice for acquiring signal, which is to avoid job quitting for single-channel collapse.

28. A data acquisition system for a solid-state urea biosensor according to claim 24, wherein the location of the acquired data in storage is a computer hard disk, a flash disk, a portable hard disk, or a network hard disk.

29. A data acquisition system for a solid-state urea biosensor according to claim 24, wherein the time interval of acquisition is dependent on the reaction time of the solid-state urea biosensor.

30. A data acquisition system for a solid-state urea biosensor according to claim 16, wherein the analyte concentration display panel shows an output potential, a concentration of urea and a plurality of warning lamps.

31. A data acquisition system for a solid-state urea biosensor according to claim 30, wherein the output potential is the actual potential outputted by the solid-state urea biosensor.

32. A data acquisition system for a solid-state urea biosensor according to claim 30, wherein the unit for the concentration of urea is either mg/100 ml or molarity.

33. A data acquisition system for a solid-state urea biosensor according to claim 30, wherein the plurality of warning lamps comprises a too-high lamp, a normal lamp, and a too-low lamp, for signaling to the operator a comparison between the analyte and clinical values, wherein if a sensed concentration is located within a too-high range, a normal range, or a too-low range, then a corresponding warning lamp lightens; if a sensed concentration is located in between the too-high range and the normal range, then the too-high lamp and the normal lamp lighten; and if a sensed concentration is located in between the normal range and the too-low range, then the normal lamp and the too-low lamp lighten.

34. A data acquisition system for a solid-state urea biosensor according to claim 33, wherein the too-high range covers concentrations of urea higher than 39 mg/dl.

35. A data acquisition system for a solid-state urea biosensor according to claim 33, wherein the normal range covers concentrations between 15-40 mg/dl.

36. A data acquisition system for a solid-state urea biosensor according to claim 33, wherein the too-low range covers concentrations lower than 16 mg/dl.
